(19) United States
(12) Patent Application Publication
(10) Pub. No.: US 2013/0115201 A1
(43) Pub. Date: May 9, 2013

(54) Fluid Supplement with Prenatal Vitamins

(71) Applicant: Michael Patrick Gleeson, San Clemente, CA (US)

(72) Inventor: Michael Patrick Gleeson, San Clemente, CA (US)

(21) Appl. No.: 13/288,382

(22) Filed: Nov. 3, 2011

(51) Int. Cl. A61K 31/714; A61P 3/00; A61P 1/08

(52) U.S. Cl. 424/94.1; 514/52

(57) ABSTRACT

A fluid supplement is provided that is specifically tailored to meet the health and nutritional needs of a pregnant woman and her developing fetus. The fluid supplement also combats the symptoms of nausea and vomiting that are commonly associated with pregnancy. The fluid supplement contains vitamins, minerals, dietary supplements, and flavor-enhancing ingredients in amounts that safely provide health and nutritional benefits to both the woman and her developing fetus. By using the fluid supplement, a pregnant woman is able to obtain the health and nutritional benefits in a single dose of a palatable fluid supplement instead of having to swallow multiple pills per day.

### FIELD OF THE INVENTION

The present invention pertains generally to fluid supplements. More particularly, the present invention pertains to fluid supplements that combat nausea and vomiting associated with pregnancy. The present invention is particularly, but not exclusively, useful as a fluid supplement for providing essential vitamins to pregnant women and their developing fetuses.

### BACKGROUND OF THE INVENTION

During pregnancy, the body of a pregnant woman undergoes significant hormonal and physiological changes. These changes are necessary to provide a proper environment for a developing fetus.

Unfortunately, many of these changes result in considerable discomfort for the prospective mother. Among the most prevalent discomforts a pregnant woman will experience are nausea and vomiting. Various medications are readily available to deal with nausea and vomiting, but most of these medications can be dangerous to the developing fetus. In most instances, a pregnant woman cannot effectively deal with symptoms, like nausea and vomiting, because of the danger to her developing fetus. Further, swallowing a pill may also be difficult or cause additional discomfort. In addition to physical discomfort, the physiological changes may also prevent vital nutrients, minerals, and vitamins from reaching the developing fetus.

Throughout a pregnancy, the pregnant woman also should follow strict dietary guidelines, and she should incorporate vitamins and minerals to counteract the effects of the physiological changes discussed above. As is commonly known, anything a pregnant woman ingests could eventually reach the developing fetus. And, many substances that are safe for adult consumption can have dire, irreversible effects on the fetus. Furthermore, the fetus relies on its mother for nourishment and proper development. Certain vitamins, like folic acid, greatly aid in proper fetal development. Many of these vitamins can be unpleasant for the mother to ingest, especially in the types of quantities needed for proper development of her fetus.

In light of the above, it is an object of the present invention to provide a remedy to combat nausea and vomiting caused by pregnancy. Another object of the present invention is to provide a fluid supplement that provides essential vitamins and nutrients to both the mother and the developing fetus. A further object of the present invention is to provide a fluid supplement that is palatable to the mother and easy to consume. Yet another object of the present invention is to provide a fluid supplement that is easy to use, is relatively simple to manufacture, and is relatively cost effective.

### SUMMARY OF THE INVENTION

In accordance with the present invention, a fluid supplement is provided for consumption by a pregnant woman. Specifically, the fluid supplement will combat the symptoms of nausea and vomiting. In addition, the fluid supplement includes vitamins, minerals, and nutritional supplements essential to the health of both the pregnant female and her developing fetus. And, several flavor-enhancing ingredients are added specifically to improve the palatability of the fluid supplement. Importantly, the fluid supplement of the present invention is safe for ingestion by both the mother and the developing fetus.

Four ingredients are of great importance to the fluid supplement of the present invention. These are: (1) folic acid, (2) calcium, (3) Vitamin D, and (4) iron. Folic acid reduces the risk of spinal cord abnormalities in the developing fetus and is essential for proper neural development. In addition, calcium and Vitamin D both aid in the development of fetal bone and tissue. Moreover, calcium assists the mother in maintaining her bone density at a time when calcium is being consumed in significant amounts by the developing fetus. Iron is included for its ability to transport oxygen via the bloodstream and for preventing maternal anemia.

Additional ingredients are also added to the four essential ingredients disclosed in the previous paragraph. These ingredients can be grouped into vitamins, minerals, nutritional supplements, and flavor-enhancing ingredients.

A first vitamin added to the fluid supplement is Vitamin C which is added to the fluid supplement because pregnant women require more Vitamin

C than the average adult. As is well-known, Vitamin C is the best vitamin for strengthening the immune system. Here, Vitamin C strengthens the immune system of both the mother and the developing fetus. Additionally, Vitamin E is added in a small amount for its antioxidant properties. Also, thiamin (Vitamin B1) is included to assist both mother and fetus with converting carbohydrates to energy and to aid the brain development of the fetus. A further vitamin included in the fluid supplement is niacin (Vitamin B3), which is included to help build the placenta and to help regulate digestion for the mother. For the developing fetus, niacin provides energy for development. Riboflavin (Vitamin B2) is included to assist in the growth of the bones, muscles, and nervous system of the fetus. Further, riboflavin protects the fetus from various post-birth health problems, like anemia. Vitamin B12 is provided to assist both mother and fetus in producing red blood cells and to assist in the development of the brain and nervous system of the fetus. A final vitamin included for the present invention is Vitamin B6, which is included to help mother and fetus produce red blood cells and to aid in the development of the brain and nervous system of the developing fetus.

Regarding minerals, Zinc is provided in the fluid supplement because of its ability to promote cell division in the fetus. As can be appreciated, cell division is responsible for the development of tissue and organ growth in the fetus. Zinc also assists both mother and fetus in developing various enzymes, like insulin. Next, potassium is added to the fluid supplement to help the mother maintain fluid and electrolyte balance, which are both adversely affected by pregnancy. The final mineral added to the present invention is magnesium. Magnesium is added to help build and repair tissue in the body of the mother. By adding magnesium, problems commonly caused by a magnesium deficiency, like preeclampsia and infant mortality, can be avoided.

Several dietary supplements are also provided for the fluid supplement of the present invention. It is commonly known that ginger is a natural digestive aid and can alleviate the symptoms associated with nausea. For this reason, ginger is included to aid digestion and to treat symptoms associated with pregnancy-related nausea and vomiting. Ginger is included in a significant quantity because of its safety and absence of any serious side effects. Also, it is well-known that Omega-3 fatty acids have significant health benefits. Yet, many people fail to get enough Omega-3 fatty acids in their diets. For this reason, two different Omega-3 fatty acids are also included for the present invention. Specifically, docosahexaenoic acid (DHA) and eicosapentaenoic acid (EPA) are the two Omega-3 fatty acids included in the fluid supplement in the form of fish oils. For a pregnant woman in particular, fatty acids lower the risk of pre-term labor, preeclampsia, and other complications related to pregnancy. Also, DHA and EPA assist in neural and visual development of the fetus in addition to maintaining the neural and visual health of the mother.

Finally, flavor-enhancing ingredients are added to improve the taste and palatability of the fluid supplement. Among these ingredients are sugars, such as dextrose and fructose, citric acid, and other natural flavoring.

To produce the fluid supplement of the present invention, all of the ingredients mentioned above are added to water. With the exception of DHA and possibly EPA, all of the ingredients are granular and will dissolve in water. DHA and EPA are commonly found in fish oil that is usually liquid in form. If added in liquid form, both will still mix easily with the water. No particular order is envisioned for the addition of the ingredients because no chemical reactions occur that will change the chemical properties of the ingredients. Only physical changes of the ingredients will take place.

By combining all of these ingredients, a pregnant woman has access to a plethora of beneficial vitamins, minerals, and nutritional supplements in one serving of a single, palatable fluid supplement. Instead of having to take several different pills, a pregnant woman can simply drink one serving of the fluid supplement per day. In doing so, both the woman and her developing fetus will be healthy.

### DESCRIPTION OF THE PREFERRED EMBODIMENTS

For the present invention, specific ingredients in specific ranges are included to meet the objectives disclosed previously. In using specific ingredients and specific amounts, the optimal health of both the mother and the developing fetus is promoted. In general, each of the ingredients added is a vitamin, a mineral, a nutritional supplement, or a flavor-enhancing ingredient. Each of the ingredients, with the exception of water, sugar, citric acid, and natural flavoring, is provided for a specific reason to benefit the health and well-being of the mother, the developing fetus, or both.

Typically, all of the following ingredients are included in the following amounts to produce the fluid supplement of the present invention. The ingredients listed below do not include the water. The ingredients listed below are all mixed with water to produce the preferred embodiment of the fluid supplement of the present invention. Serving size for the fluid supplement is envisioned to be between approximately 355 milliliters (12 fluid ounces) and 710 milliliters (24 fluid ounces). The high and low percentages are based on a baseline, ideal amount of the disclosed ingredients and may vary. In Table A, all ingredients have been converted to milligrams.

TABLE A
Preferred Embodiment

| | | Range | Range | | |
|---|---|---|---|---|---|
| | | Amount | Amount | | |
| | | Low | High | | |
| | | (milli- | (milli- | | |
| Item # | Ingredient | grams) | grams) | Low % | High % |
| 1 | Folic Acid | 0.72 | 0.88 | 0.090 | 0.110 |
| 2 | Calcium | 48 | 72 | 6.014 | 9.021 |
| 3 | Vitamin D | 0.009 | 0.011 | 0.001 | 0.002 |
| 4 | Iron | 7.8 | 10.2 | 0.977 | 1.278 |
| 5 | Vitamin C | 180 | 220 | 22.552 | 27.564 |
| 6 | Zinc | 4 | 6 | 0.501 | 0.752 |
| 7 | Vitamin E | 10.7 | 15 | 1.341 | 01.879 |
| 8 | Thiamine | 2.7 | 3.3 | 0.338 | 0.413 |
| 9 | Niacin | 18 | 22 | 2.255 | 2.756 |
| 10 | Riboflavin | 1.8 | 2.2 | 0.226 | 0.276 |
| 11 | Vitamin | 0.0054 | 0.0066 | 0.001 | 0.002 |
| | B-12 | | | | |
| 12 | Vitamin | 22.5 | 27.5 | 2.819 | 3.445 |
| | B-6 | | | | |
| 13 | DHA | Varies | Varies | Varies | Varies |
| 14 | EPA | Varies | Varies | Varies | Varies |
| 15 | Ginger | 225 | 275 | 28.190 | 34.445 |
| | extract | | | | |
| 16 | Sodium | 108 | 132 | 13.531 | 16.538 |
| | Chloride | | | | |
| 17 | Potassium | 27 | 33 | 3.383 | 4.135 |
| 18 | Magnesium | 48 | 72 | 6.014 | 9.021 |
| 19 | Dextrose | Varies | Varies | Varies | Varies |
| 20 | Fructose | Varies | Varies | Varies | Varies |
| 21 | Citric Acid | Varies | Varies | Varies | Varies |
| 22 | Natural | Varies | Varies | Varies | Varies |
| | Flavors | | | | |

While the particular Fluid Supplement with Prenatal Vitamins as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A fluid composition for supplementing the diet during pregnancy which comprises effective amounts of folic acid, calcium, Vitamin D, iron, Vitamin C, zinc, Vitamin E, thiamin, niacin, riboflavin, Vitamin B-12, Vitamin B-6, docosahexaenoic acid (DHA), eicosapentaenoic acid (EPA), ginger extract, sodium chloride, potassium, magnesium, dextrose, fructose, citric acid, and natural flavors to combat symptoms of nausea and vomiting and to provide a pregnant mother and a developing fetus with essential vitamins.

2. A fluid composition as recited in claim 1 wherein the docosahexaenoic acid (DHA) and eicosapentaenoic acid (EPA) are in liquid form.

3. A fluid composition as recited in claim 1 wherein the folic acid, calcium, Vitamin D, iron, Vitamin C, zinc, Vitamin E, thiamin, niacin, riboflavin, Vitamin B-12, Vitamin B-6, ginger extract, sodium chloride, potassium, magnesium, dextrose, fructose, citric acid, and natural flavors are in granular form.

4. A fluid composition as recited in claim 1 further comprising a predetermined amount of water.

5. A fluid composition as recited in claim 4 wherein the ingredients are mixed with the predetermined amount of water to produce the fluid composition.

6. A fluid composition for supplementing the diet during pregnancy which comprises the following ingredients and ranges:
720-880 mcg of folic acid;
48-72 mg of calcium;
360-440 IU of Vitamin D;
7.8-10.2 mg of iron;
180-220 mg of Vitamin C;
4-6 mg of zinc;
10.7-15 mg of Vitamin E;
2.7-3.3 mg of thiamin;
18-22 mg of niacin;
1.8-2.2 mg of riboflavin;
5.4-6.6 mcg of Vitamin B-12;
22.5-27.5 mg of Vitamin B-6;
225-275 mg of ginger extract;
108-132 mg of sodium chloride (NaCl);
27-33 mg of potassium;
48-72 mg of magnesium;
docosahexaenoic acid (DHA);
eicosapentaenoic acid (EPA);
dextrose;
fructose;
citric acid; and
natural flavors.

7. A fluid composition as recited in claim 6 further comprising a predetermined amount of water, wherein the ingredients are combined with the predetermined amount of water to produce the fluid supplement.

8. A fluid composition as recited in claim 7 wherein the docosahexaenoic acid (DHA) and eicosapentaenoic acid (EPA) are in liquid form prior to combining the ingredients with the predetermined amount of water.

9. A fluid composition as recited in claim 8 wherein the folic acid, calcium, Vitamin D, iron, Vitamin C, zinc, Vitamin E, thiamine, niacin, riboflavin, Vitamin B-12, Vitamin B-6, ginger extract, sodium chloride, potassium, magnesium, dextrose, fructose, citric acid, and natural flavors are in granular form prior to combining the ingredients with the predetermined amount of water.

10. A fluid composition for supplementing the diet during pregnancy which comprises the following ingredients and ranges by percent weight:
Folic acid: 0.090-0.110%;
Calcium: 6.014-9.021%;
Vitamin D: 0.001-0.002%;
Iron: 0.977-1.278%;
Vitamin C: 22.552-27.564%;
Zinc: 0.501-0.752%;
Vitamin E: 1.341-1.879%;
Thiamine: 0.338-0.413%;
Niacin: 2.255-2.756%;
Riboflavin: 0.226-0.276%;
Vitamin B12: 0.001-0.002%;
Vitamin B6: 2.819-3.445%;
Ginger extract: 28.190-34.455%;
Sodium Chloride (NaCl): 13.531-16.538%;
Potassium: 3.383-4.135%; and
Magnesium: 6.014-9.021%.

11. A fluid composition as recited in claim 10 further comprising dextrose, wherein the dextrose is added to increase palatability.

12. A fluid composition as recited in claim 11 further comprising fructose, wherein the fructose is added to increase palatability.

13. A fluid composition as recited in claim 12 further comprising eicosapentaenoic acid (EPA) and docosahexaenoic acid (DHA).

14. A fluid composition as recited in claim 13 further comprising natural flavoring, wherein the natural flavoring is added to increase palatability.

15. A fluid composition as recited in claim 14 further comprising citric acid, wherein the citric acid is added to increase palatability.

16. A fluid composition as recited in claim 15 wherein the ingredients are mixed with a predetermined amount of water to produce the fluid supplement.

17. A fluid composition as recited in claim 16 wherein the folic acid, calcium, Vitamin D, iron, Vitamin C, zinc, Vitamin E, thiamine, niacin, riboflavin, Vitamin B-12, Vitamin B-6, ginger extract, sodium chloride, potassium, magnesium, dextrose, fructose, citric acid, and natural flavoring are in granular form prior to being combined with the predetermined amount of water.

18. A fluid composition as recited in claim 17 wherein the docosahexaenoic acid (DHA) and eicosapentaenoic acid (EPA) are in liquid form prior to being combined with the predetermined amount of water.
